SEQUENCE LISTING (18 sequences)

SEQ ID NO: 1 - DNA, 2053 bp, Musa acuminata
  source 1..2053: mol_type = genomic DNA
atggcttctatctctcagctaatcactacaagcatccccaccacttcatgccccttctcccctccgagaaccacggtatcgatctccggctccaaacaccaccaccgcgtttcccccatctcatgctccaccagagaccacaaccaacccctcgtcgatcccacagtcgaccgccgccacgtcctcgtcggactaggcagcctctacggcgcctccgctgcactaacgtccctccgcgaggccagtgcggcaccgatcgcggcgcccgacctgtccgcatgcgggccggctgacctccctccggatgccactccgacgaactgctgcccgccgtccgcgggcgacgcgaccgagttcgtcattcccgacccgtcctcgcccttgagggtgcgcccggcggcccactcggtcgacaaagattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgacgacagcttcgcgattccgttctggaactgggacgcccctgacgggatgcgattgccagcgatgtacgtggatcccacgtcgccgctttacgatcccctaagggatgcacagcatcagccgccgacgttagtggatttggacttcggagggatcgatccttctttcagtgataagcagcagattgatcacaacctcaaggttatgtacaggcaggtaattaatggactccaataccactgaatcagtcatgcattatgttattgtctgtacgagcatcataaaataatgattgataggcgacatcagccataattatcctgcattaattgacgaatcttgtaaacaatgtcatcttgttgggctttccatggctcatggagttgattgatgtgattctacgtcaatgatcgcttggagaacgtaacttaagcttgatgggtcaaacttcttgtctcgatgacgacagaatactgtcttttgatgcttagttttgtgtcatattaactgcaagatttttctgcagatcgtctcgaatgcaccgacaccgaggctcttcttcggaaacccctaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttctttgcccaccactccaacatcgaccgcatctggaacgtgtggaagggcctcggtggccggcgcaaggacctggccgaccccgactggctcgacgcctccttcgtcttctacgacgagaacgccaacctcgtcaagatccgagttcgcgactgcatcgactcagacaagctacgctacgagtaccaggacgtcggtaacccatggctcaacacacgcccgacggtgacgtccggagtgaggccgagagtggccggagtggcgcatgcaaacgtcgtggagccgaagtttccgataaagttggactcggtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgaccacgggaaggtcgggccggggggccgggagctcgccgggagcttcgtgaacgtgcctcacaggcacaagcatgacaagatgagcaagctgctgaagaccaggctgcagctgggcttgactgagctgttggaggatctcaaggctgaaggagatgggagcatcatggtgactttggtgccgaggcaggggaaggggaaggtgaaggttggtagtctcaagatcgagttagttgattga

SEQ ID NO: 2 - DNA, 2068 bp, Musa acuminata
  source 1..2068: mol_type = genomic DNA
atggcttctatctcgcagctaatcactacaagcatccccaccacctttcctctctcctcttcatgccccttctcccctccgagaaccacggtatcgatctccggcttcaaacaccaccaccacgtttcccccatctcatgctcctccagagaccacaaccaacccctcgttgatcccacagtcgatcgccgccacgtcctcgtcggactaggcagcctctacggcgcctccgctgctctaacgtccctccacgaggccagcgcggcaccgatcgcggcgcccgacctgtccgcatgcgggccggctgacctccctccggatgccactccgacgaactgctgcctgccgtccgcgggcgacgcgaccgagttcgtcattcccgacccgtcctcgcccttgagggtgcgcccggcggcccactcggtcgacaacgattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgacgacagcttcgcgattccgttctggaactgggacgcccctgacgggatgcgattgccagcgatgtacgtggatcccacgtcgccgctttacgatcccctaagggatgcacagcatcagccgccgacgttggtggatttggacttcggagggatcgatccttctttcagtgataagcagcagattgatcacaacctcaaggttatgtacaggcaggtaattaatggactccaataccactgaatcagtcatgcattatgttattatttgtacgagcatcataaaatgatgattgataggcgacatcagccataattatcctgcattaattgacgaatcttgtaaacaatgtcatcttgttgggctttccatggctcatggagttgattgatgtgattctacgtcaatgatcgcttggagaacgtaacttaagcttgatgggtcaaacttcttgtctcgatgacgacagaatactgtcttttgatgcttagttttgtgtaatattaactgcaagatttttctgcagatcgtctcgaatgcaccgacaccgaggctcttcttcggaaacccgtaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttcttcgcccaccactccaacatcgaccgcatctggaacgtgtggaagggtctcggtagccggcgcaaggacctggccgaccccgactggctcgacgcctccttcgtcttctacgacgagaacgccaacctcgtcaagatccgagttcgcgactgcatcgactcagacaagctacgctacgagtaccaggacgtcggtaacctatggctcaacacacgcccgacggtgacgtccggagtgaggccgagagtggccggagtggcgcatgcaaacgtggtggagccgaagtttccgataaagttggactcggtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgatcacgggaaggtcgggccggggggccgggagctcgccgggagcttcgtgaacgtgcctcacaggcacaagcatgacaagatgagcaagcagctgaagaccaggctgcagctgggcttgactgagctgttggaggatctcaaggttgaaggagatgggagcatcatggtgactttggtgccgaggcaggggaaggggaaggtgaaggttagcagtctcaagatcgagttagttgattga

SEQ ID NO: 3 - DNA, 2068 bp, Musa acuminata
  source 1..2068: mol_type = genomic DNA
atggcttctatctcgcagctaatcactacaagcatccccaccaccttttctctctcctattcatgccccttctcccctccgagaaccacggtatcgatctccggctccaaacaccaccaccacgtttcccccatctcatgctccaccagagaccacaaccaacccctcgtcgatcccacagtcgaccgccgccacgtcctcgtcggactaggcagcctctacggcgcctccgctgcactaacgtccctccgcgaggccagtgcggcaccgatcgcggcgcccgacctgtccgcatgcgggccggctgacctccctccggatgccactccgacgaactgctgcccgccgtccgcgggcgacgcgaccgagttcgtcattcccgacccgtcctcgcccttgagggtgcgcccggcggcccactcggtcgacaaagattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgatgacagcttcgcgattccgttctggaactgggacgcccctgacgggatgcgattgccagcgatgtacgtggatcccacgtcgccgctttacgatcccctaagggatgcacagcatcagccgccgacgttagtggatttggacttcggagggatggatccttctttcagtgataagcagcagattgatcacaacctcaaggttatgtacaggcaggtaattaatggactccaaaaccactgaatcagtcatgcattatgttattatttgtacgagcatcataaaataatgattgataggcgacatcagccataattatcctgcattaattgacgaatcttgtaaacaatgacatcttgttgggctttccatggctcatggagttgattgatgtgattctacgtcaatgatcgcttggagaacgtaacttaagcttgatgggtcaaacttcttgtctcgatgacgacagaatactgtcttttgatgcttagttttgtgtcatattaactgcaagatttttctgcagatcgtctcgaatgcaccgacaccgaggctcttcttcggaaacccctaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttctttgcccaccactccaacatcgaccgcatctggaacgtgtggaagggtctcggtagccggcgcaaggacctggccgaccccgactggctcgacgcctccttcgtcttctacgacgagaacgccaacctcgtcaagatccgaattcgcgactgcatcgactcagacaagctacgctacgagtaccaggacgtcggtaacccatggctcaacacacgcccgacggtgacgtccggagtgaggccgagagtggccggagtggcgcatgcaaacgtcgtggagccgaagtttccgataaagttggactcggtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgaccacgggaaggtcgggccggggggccgggagctcgccgggagcttcgtgaacgtgcctcacaggcacaagcatgacaagatgagcaagcggctgaagaccaggctgcagctgggcttgactgagctgttggaggatctcaaggctgaaggagatgggagcatcatggtgactttggtgccgaggcaggggaaggggaaggtgaaggttggcagtctcaagatcgagttagttgattga

SEQ ID NO: 4 - DNA, 1743 bp, Musa acuminata
  source 1..1743: mol_type = genomic DNA
atggcttctatctctcagctaatcactacaagcatccccaccacttcatgccccttctcccctccgagaaccacggtatcgatctccggctccaaacaccaccaccgcgtttcccccatctcatgctccaccagagaccacaaccaacccctcgtcgatcccacagtcgaccgccgccacgtcctcgtcggactaggcagcctctacggcgcctccgctgcactaacgtccctccgcgaggccagtgcggcaccgatcgcggcgcccgacctgtccgcatgcgggccggctgacctccctccggatgccactccgacgaactgctgcccgccgtccgcgggcgacgcgaccgagttcgtcattcccgacccgtcctcgcccttgagggtgcgcccggcggcccactcggtcgacaaagattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgacgacagcttcgcgattccgttctggaactgggacgcccctgacgggatgcgattgccagcgatgtacgtggatcccacgtcgccgctttacgatcccctaagggatgcacagcatcagccgccgacgttagtggatttggacttcggagggatcgatccttctttcagtgataagcagcagattgatcacaacctcaaggttatgtacaggcagatcgtctcgaatgcaccgacaccgaggctcttcttcggaaacccctaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttctttgcccaccactccaacatcgaccgcatctggaacgtgtggaagggcctcggtggccggcgcaaggacctggccgaccccgactggctcgacgcctccttcgtcttctacgacgagaacgccaacctcgtcaagatccgagttcgcgactgcatcgactcagacaagctacgctacgagtaccaggacgtcggtaacccatggctcaacacacgcccgacggtgacgtccggagtgaggccgagagtggccggagtggcgcatgcaaacgtcgtggagccgaagtttccgataaagttggactcggtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgaccacgggaaggtcgggccggggggccgggagctcgccgggagcttcgtgaacgtgcctcacaggcacaagcatgacaagatgagcaagctgctgaagaccaggctgcagctgggcttgactgagctgttggaggatctcaaggctgaaggagatgggagcatcatggtgactttggtgccgaggcaggggaaggggaaggtgaaggttggtagtctcaagatcgagttagttgattga

SEQ ID NO: 5 - DNA, 1758 bp, Musa acuminata
  source 1..1758: mol_type = genomic DNA
atggcttctatctcgcagctaatcactacaagcatccccaccacctttcctctctcctcttcatgccccttctcccctccgagaaccacggtatcgatctccggcttcaaacaccaccaccacgtttcccccatctcatgctcctccagagaccacaaccaacccctcgttgatcccacagtcgatcgccgccacgtcctcgtcggactaggcagcctctacggcgcctccgctgctctaacgtccctccacgaggccagcgcggcaccgatcgcggcgcccgacctgtccgcatgcgggccggctgacctccctccggatgccactccgacgaactgctgcctgccgtccgcgggcgacgcgaccgagttcgtcattcccgacccgtcctcgcccttgagggtgcgcccggcggcccactcggtcgacaacgattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgacgacagcttcgcgattccgttctggaactgggacgcccctgacgggatgcgattgccagcgatgtacgtggatcccacgtcgccgctttacgatcccctaagggatgcacagcatcagccgccgacgttggtggatttggacttcggagggatcgatccttctttcagtgataagcagcagattgatcacaacctcaaggttatgtacaggcagatcgtctcgaatgcaccgacaccgaggctcttcttcggaaacccgtaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttcttcgcccaccactccaacatcgaccgcatctggaacgtgtggaagggtctcggtagccggcgcaaggacctggccgaccccgactggctcgacgcctccttcgtcttctacgacgagaacgccaacctcgtcaagatccgagttcgcgactgcatcgactcagacaagctacgctacgagtaccaggacgtcggtaacctatggctcaacacacgcccgacggtgacgtccggagtgaggccgagagtggccggagtggcgcatgcaaacgtggtggagccgaagtttccgataaagttggactcggtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgatcacgggaaggtcgggccggggggccgggagctcgccgggagcttcgtgaacgtgcctcacaggcacaagcatgacaagatgagcaagcagctgaagaccaggctgcagctgggcttgactgagctgttggaggatctcaaggttgaaggagatgggagcatcatggtgactttggtgccgaggcaggggaaggggaaggtgaaggttagcagtctcaagatcgagttagttgattga

SEQ ID NO: 6 - DNA, 1758 bp, Musa acuminata
  source 1..1758: mol_type = genomic DNA
atggcttctatctcgcagctaatcactacaagcatccccaccaccttttctctctcctattcatgccccttctcccctccgagaaccacggtatcgatctccggctccaaacaccaccaccacgtttcccccatctcatgctccaccagagaccacaaccaacccctcgtcgatcccacagtcgaccgccgccacgtcctcgtcggactaggcagcctctacggcgcctccgctgcactaacgtccctccgcgaggccagtgcggcaccgatcgcggcgcccgacctgtccgcatgcgggccggctgacctccctccggatgccactccgacgaactgctgcccgccgtccgcgggcgacgcgaccgagttcgtcattcccgacccgtcctcgcccttgagggtgcgcccggcggcccactcggtcgacaaagattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgatgacagcttcgcgattccgttctggaactgggacgcccctgacgggatgcgattgccagcgatgtacgtggatcccacgtcgccgctttacgatcccctaagggatgcacagcatcagccgccgacgttagtggatttggacttcggagggatggatccttctttcagtgataagcagcagattgatcacaacctcaaggttatgtacaggcagatcgtctcgaatgcaccgacaccgaggctcttcttcggaaacccctaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttctttgcccaccactccaacatcgaccgcatctggaacgtgtggaagggtctcggtagccggcgcaaggacctggccgaccccgactggctcgacgcctccttcgtcttctacgacgagaacgccaacctcgtcaagatccgaattcgcgactgcatcgactcagacaagctacgctacgagtaccaggacgtcggtaacccatggctcaacacacgcccgacggtgacgtccggagtgaggccgagagtggccggagtggcgcatgcaaacgtcgtggagccgaagtttccgataaagttggactcggtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgaccacgggaaggtcgggccggggggccgggagctcgccgggagcttcgtgaacgtgcctcacaggcacaagcatgacaagatgagcaagcggctgaagaccaggctgcagctgggcttgactgagctgttggaggatctcaaggctgaaggagatgggagcatcatggtgactttggtgccgaggcaggggaaggggaaggtgaaggttggcagtctcaagatcgagttagttgattga

SEQ ID NO: 7 - protein, 580 aa, Musa acuminata
MASISQLITTSIPTTSCPFSPPRTTVSISGSKHHHRVSPISCSTRDHNQPLVDPTVDRRHVLVGLGSLYGASAALTSLREASAAPIAAPDLSACGPADLPPDATPTNCCPPSAGDATEFVIPDPSSPLRVRPAAHSVDKDYIAKFAKGVALMKALPADDPRNFTQHANVHCAYCDGAYSQVGFPDLELQVHNSWLFLPWHRCYLYFFERILGKLIGDDSFAIPFWNWDAPDGMRLPAMYVDPTSPLYDPLRDAQHQPPTLVDLDFGGIDPSFSDKQQIDHNLKVMYRQIVSNAPTPRLFFGNPYRAGDNPNPGGGSLENVPHGPVHVWTGDRSQSELEDMGNLYSAARDPVFFAHHSNIDRIWNVWKGLGGRRKDLADPDWLDASFVFYDENANLVKIRVRDCIDSDKLRYEYQDVGNPWLNTRPTVTSGVRPRVAGVAHANVVEPKFPIKLDSVVTAKVKRPKAARTKEEKEEKEEVLVVEGIELDRDVHVKFDVFVNVTDHGKVGPGGRELAGSFVNVPHRHKHDKMSKLLKTRLQLGLTELLEDLKAEGDGSIMVTLVPRQGKGKVKVGSLKIELVD

SEQ ID NO: 8 - protein, 585 aa, Musa acuminata
MASISQLITTSIPTTFPLSSSCPFSPPRTTVSISGFKHHHHVSPISCSSRDHNQPLVDPTVDRRHVLVGLGSLYGASAALTSLHEASAAPIAAPDLSACGPADLPPDATPTNCCLPSAGDATEFVIPDPSSPLRVRPAAHSVDNDYIAKFAKGVALMKALPADDPRNFTQHANVHCAYCDGAYSQVGFPDLELQVHNSWLFLPWHRCYLYFFERILGKLIGDDSFAIPFWNWDAPDGMRLPAMYVDPTSPLYDPLRDAQHQPPTLVDLDFGGIDPSFSDKQQIDHNLKVMYRQIVSNAPTPRLFFGNPYRAGDNPNPGGGSLENVPHGPVHVWTGDRSQSELEDMGNLYSAARDPVFFAHHSNIDRIWNVWKGLGSRRKDLADPDWLDASFVFYDENANLVKIRVRDCIDSDKLRYEYQDVGNLWLNTRPTVTSGVRPRVAGVAHANVVEPKFPIKLDSVVTAKVKRPKAARTKEEKEEKEEVLVVEGIELDRDVHVKFDVFVNVTDHGKVGPGGRELAGSFVNVPHRHKHDKMSKQLKTRLQLGLTELLEDLKVEGDGSIMVTLVPRQGKGKVKVSSLKIELVD

SEQ ID NO: 9 - protein, 585 aa, Musa acuminata
MASISQLITTSIPTTFSLSYSCPFSPPRTTVSISGSKHHHHVSPISCSTRDHNQPLVDPTVDRRHVLVGLGSLYGASAALTSLREASAAPIAAPDLSACGPADLPPDATPTNCCPPSAGDATEFVIPDPSSPLRVRPAAHSVDKDYIAKFAKGVALMKALPADDPRNFTQHANVHCAYCDGAYSQVGFPDLELQVHNSWLFLPWHRCYLYFFERILGKLIGDDSFAIPFWNWDAPDGMRLPAMYVDPTSPLYDPLRDAQHQPPTLVDLDFGGMDPSFSDKQQIDHNLKVMYRQIVSNAPTPRLFFGNPYRAGDNPNPGGGSLENVPHGPVHVWTGDRSQSELEDMGNLYSAARDPVFFAHHSNIDRIWNVWKGLGSRRKDLADPDWLDASFVFYDENANLVKIRIRDCIDSDKLRYEYQDVGNPWLNTRPTVTSGVRPRVAGVAHANVVEPKFPIKLDSVVTAKVKRPKAARTKEEKEEKEEVLVVEGIELDRDVHVKFDVFVNVTDHGKVGPGGRELAGSFVNVPHRHKHDKMSKRLKTRLQLGLTELLEDLKAEGDGSIMVTLVPRQGKGKVKVGSLKIELVD

SEQ ID NO: 10 - DNA, 2054 bp, synthetic construct
  source 1..2054: mol_type = other DNA
atggcttctatctctcagctaatcactacaagcatccccaccacttcatgccccttctcccctccgagaaccacggtatcgatctccggctccaaacaccaccaccgcgtttcccccatctcatgctccaccagagaaccacaaccaacccctcgtcgatcccacagtcgaccgccgccacgtcctcgtcggactaggcagcctctacggcgcctccgctgcactaacgtccctccgcgaggccagtgcggcaccgatcgcggcgcccgacctgtccgcatgcgggccggctgacctccctccggatgccactccgacgaactgctgcccgccgtccgcgggcgacgcgaccgagttcgtcattcccgacccgtcctcgcccttgagggtgcgcccggcggcccactcggtcgacaaagattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgacgacagcttcgcgattccgttctggaactgggacgcccctgacgggatgcgattgccagcgatgtacgtggatcccacgtcgccgctttacgatcccctaagggatgcacagcatcagccgccgacgttagtggatttggacttcggagggatcgatccttctttcagtgataagcagcagattgatcacaacctcaaggttatgtacaggcaggtaattaatggactccaataccactgaatcagtcatgcattatgttattgtctgtacgagcatcataaaataatgattgataggcgacatcagccataattatcctgcattaattgacgaatcttgtaaacaatgtcatcttgttgggctttccatggctcatggagttgattgatgtgattctacgtcaatgatcgcttggagaacgtaacttaagcttgatgggtcaaacttcttgtctcgatgacgacagaatactgtcttttgatgcttagttttgtgtcatattaactgcaagatttttctgcagatcgtctcgaatgcaccgacaccgaggctcttcttcggaaacccctaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttctttgcccaccactccaacatcgaccgcatctggaacgtgtggaagggcctcggtggccggcgcaaggacctggccgaccccgactggctcgacgcctccttcgtcttctacgacgagaacgccaacctcgtcaagatccgagttcgcgactgcatcgactcagacaagctacgctacgagtaccaggacgtcggtaacccatggctcaacacacgcccgacggtgacgtccggagtgaggccgagagtggccggagtggcgcatgcaaacgtcgtggagccgaagtttccgataaagttggactcggtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgaccacgggaaggtcgggccggggggccgggagctcgccgggagcttcgtgaacgtgcctcacaggcacaagcatgacaagatgagcaagctgctgaagaccaggctgcagctgggcttgactgagctgttggaggatctcaaggctgaaggagatgggagcatcatggtgactttggtgccgaggcaggggaaggggaaggtgaaggttggtagtctcaagatcgagttagttgattga

SEQ ID NO: 11 - DNA, 2060 bp, synthetic construct
  source 1..2060: mol_type = other DNA
atggcttctatctcgcagctaatcactacaagcatccccaccacctttcctctctcctcttcatgccccttctcccctccgagaaccacggtatcgatctccggcttcaaacaccaccaccacgtttcccccatctcatgctcctccacaaccaacccctcgttgatcccacagtcgatcgccgccacgtcctcgtcggactaggcagcctctacggcgcctccgctgctctaacgtccctccacgaggccagcgcggcaccgatcgcggcgcccgacctgtccgcatgcgggccggctgacctccctccggatgccactccgacgaactgctgcctgccgtccgcgggcgacgcgaccgagttcgtcattcccgaccgtcctcgcccttgagggtgcgcccggcggcccactcggtcgacaacgattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgacgacagcttcgcgattccgttctggaactgggacgcccctgacgggatgcgattgccagcgatgtacgtggatcccacgtcgccgctttacgatcccctaagggatgcacagcatcagccgccgacgttggtggatttggacttcggagggatcgatccttctttcagtgataagcagcagattgatcacaacctcaaggttatgtacaggcaggtaattaatggactccaataccactgaatcagtcatgcattatgttattatttgtacgagcatcataaaatgatgattgataggcgacatcagccataattatcctgcattaattgacgaatcttgtaaacaatgtcatcttgttgggctttccatggctcatggagttgattgatgtgattctacgtcaatgatcgcttggagaacgtaacttaagcttgatgggtcaaacttcttgtctcgatgacgacagaatactgtcttttgatgcttagttttgtgtaatattaactgcaagatttttctgcagatcgtctcgaatgcaccgacaccgaggctcttcttcggaaacccgtaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttcttcgcccaccactccaacatcgaccgcatctggaacgtgtggaagggtctcggtagccggcgcaaggacctggccgaccccgactggctcgacgcctccttcgtcttctacgacgagaacgccaacctcgtcaagatccgagttcgcgactgcatcgactcagacaagctacgctacgagtaccaggacgtcggtaacctatggctcaacacacgcccgacggtgacgtccggagtgaggccgagagtggccggagtggcgcatgcaaacgtggtggagccgaagtttccgataaagttggactcggtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgatcacgggaaggtcgggccggggggccgggagctcgccgggagcttcgtgaacgtgcctcacaggcacaagcatgacaagatgagcaagcagctgaagaccaggctgcagctgggcttgactgagctgttggaggatctcaaggttgaaggagatgggagcatcatggtgactttggtgccgaggcaggggaaggggaaggtgaaggttagcagtctcaagatcgagttagttgattga

SEQ ID NO: 12 - DNA, 2067 bp, synthetic construct
  source 1..2067: mol_type = other DNA
atggcttctatctcgcagctaatcactacaagcatccccaccaccttttctctctcctattcatgccccttctcccctccgagaaccacggtatcgatctccggctccaaacaccaccaccacgtttcccccatctcatgctccaccagagaccacaaccaacccctcgtcgatcccacagtcgaccgccgccacgtcctcgtcggactaggcagcctctacggcgcctccgctgcactaacgtccctccgcgaggccagtgcggcaccgatcgcggcgcccgacctgtccgcatgcgggccggctgacctccctccggatgccactccgacgaactgctgcccgccgtccgcgggcgacgcgaccgagttcgtcattcccgaccgtcctcgcccttgagggtgcgcccggcggcccactcggtcgacaaagattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgatgacagcttcgcgattccgttctggaactgggacgcccctgacgggatgcgattgccagcgatgtacgtggatcccacgtcgccgctttacgatcccctaagggatgcacagcatcagccgccgacgttagtggatttggacttcggagggatggatccttctttcagtgataagcagcagattgatcacaacctcaaggttatgtacaggcaggtaattaatggactccaaaaccactgaatcagtcatgcattatgttattatttgtacgagcatcataaaataatgattgataggcgacatcagccataattatcctgcattaattgacgaatcttgtaaacaatgacatcttgttgggctttccatggctcatggagttgattgatgtgattctacgtcaatgatcgcttggagaacgtaacttaagcttgatgggtcaaacttcttgtctcgatgacgacagaatactgtcttttgatgcttagttttgtgtcatattaactgcaagatttttctgcagatcgtctcgaatgcaccgacaccgaggctcttcttcggaaacccctaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttctttgcccaccactccaacatcgaccgcatctggaacgtgtggaagggtctcggtagccggcgcaaggacctggccgaccccgactggctcgacgcctccttcgtcttctacgacgagaacgccaacctcgtcaagatccgaattcgcgactgcatcgactcagacaagctacgctacgagtaccaggacgtcggtaacccatggctcaacacacgcccgacggtgacgtccggagtgaggccgagagtggccggagtggcgcatgcaaacgtcgtggagccgaagtttccgataaagttggactcggtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgaccacgggaaggtcgggccggggggccgggagctcgccgggagcttcgtgaacgtgcctcacaggcacaagcatgacaagatgagcaagcggctgaagaccaggctgcagctgggcttgactgagctgttggaggatctcaaggctgaaggagatgggagcatcatggtgactttggtgccgaggcaggggaaggggaaggtgaaggttggcagtctcaagatcgagttagttgattga

SEQ ID NO: 13 - DNA, 1744 bp, synthetic construct
  source 1..1744: mol_type = other DNA
atggcttctatctctcagctaatcactacaagcatccccaccacttcatgccccttctcccctccgagaaccacggtatcgatctccggctccaaacaccaccaccgcgtttcccccatctcatgctccaccagagaaccacaaccaacccctcgtcgatcccacagtcgaccgccgccacgtcctcgtcggactaggcagcctctacggcgcctccgctgcactaacgtccctccgcgaggccagtgcggcaccgatcgcggcgcccgacctgtccgcatgcgggccggctgacctccctccggatgccactccgacgaactgctgcccgccgtccgcgggcgacgcgaccgagttcgtcattcccgacccgtcctcgcccttgagggtgcgcccggcggcccactcggtcgacaaagattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgacgacagcttcgcgattccgttctggaactgggacgcccctgacgggatgcgattgccagcgatgtacgtggatcccacgtcgccgctttacgatcccctaagggatgcacagcatcagccgccgacgttagtggatttggacttcggagggatcgatccttctttcagtgataagcagcagattgatcacaacctcaaggttatgtacaggcagatcgtctcgaatgcaccgacaccgaggctcttcttcggaaacccctaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttctttgcccaccactccaacatcgaccgcatctggaacgtgtggaagggcctcggtggccggcgcaaggacctggccgaccccgactggctcgacgcctccttcgtcttctacgacgagaacgccaacctcgtcaagatccgagttcgcgactgcatcgactcagacaagctacgctacgagtaccaggacgtcggtaacccatggctcaacacacgcccgacggtgacgtccggagtgaggccgagagtggccggagtggcgcatgcaaacgtcgtggagccgaagtttccgataaagttggactcggtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgaccacgggaaggtcgggccggggggccgggagctcgccgggagcttcgtgaacgtgcctcacaggcacaagcatgacaagatgagcaagctgctgaagaccaggctgcagctgggcttgactgagctgttggaggatctcaaggctgaaggagatgggagcatcatggtgactttggtgccgaggcaggggaaggggaaggtgaaggttggtagtctcaagatcgagttagttgattga

SEQ ID NO: 14 - DNA, 1750 bp, synthetic construct
  source 1..1750: mol_type = other DNA
atggcttctatctcgcagctaatcactacaagcatccccaccacctttcctctctcctcttcatgccccttctcccctccgagaaccacggtatcgatctccggcttcaaacaccaccaccacgtttcccccatctcatgctcctccacaaccaacccctcgttgatcccacagtcgatcgccgccacgtcctcgtcggactaggcagcctctacggcgcctccgctgctctaacgtccctccacgaggccagcgcggcaccgatcgcggcgcccgacctgtccgcatgcgggccggctgacctccctccggatgccactccgacgaactgctgcctgccgtccgcgggcgacgcgaccgagttcgtcattcccgaccgtcctcgcccttgagggtgcgcccggcggcccactcggtcgacaacgattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgacgacagcttcgcgattccgttctggaactgggacgcccctgacgggatgcgattgccagcgatgtacgtggatcccacgtcgccgctttacgatcccctaagggatgcacagcatcagccgccgacgttggtggatttggacttcggagggatcgatccttctttcagtgataagcagcagattgatcacaacctcaaggttatgtacaggcagatcgtctcgaatgcaccgacaccgaggctcttcttcggaaacccgtaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttcttcgcccaccactccaacatcgaccgcatctggaacgtgtggaagggtctcggtagccggcgcaaggacctggccgaccccgactggctcgacgcctccttcgtcttctacgacgagaacgccaacctcgtcaagatccgagttcgcgactgcatcgactcagacaagctacgctacgagtaccaggacgtcggtaacctatggctcaacacacgcccgacggtgacgtccggagtgaggccgagagtggccggagtggcgcatgcaaacgtggtggagccgaagtttccgataaagttggactcggtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgatcacgggaaggtcgggccggggggccgggagctcgccgggagcttcgtgaacgtgcctcacaggcacaagcatgacaagatgagcaagcagctgaagaccaggctgcagctgggcttgactgagctgttggaggatctcaaggttgaaggagatgggagcatcatggtgactttggtgccgaggcaggggaaggggaaggtgaaggttagcagtctcaagatcgagttagttgattga

SEQ ID NO: 15 - DNA, 1757 bp, synthetic construct
  source 1..1757: mol_type = other DNA
atggcttctatctcgcagctaatcactacaagcatccccaccaccttttctctctcctattcatgccccttctcccctccgagaaccacggtatcgatctccggctccaaacaccaccaccacgtttcccccatctcatgctccaccagagaccacaaccaacccctcgtcgatcccacagtcgaccgccgccacgtcctcgtcggactaggcagcctctacggcgcctccgctgcactaacgtccctccgcgaggccagtgcggcaccgatcgcggcgcccgacctgtccgcatgcgggccggctgacctccctccggatgccactccgacgaactgctgcccgccgtccgcgggcgacgcgaccgagttcgtcattcccgaccgtcctcgcccttgagggtgcgcccggcggcccactcggtcgacaaagattacatagctaagttcgcgaagggcgtcgctctcatgaaggcgcttccggccgacgacccccggaacttcactcagcatgccaacgtgcactgcgcctactgcgacggggcgtacagccaagtcggcttcccggaccttgagctccaggtgcacaactcatggctcttcctgccatggcaccgctgctacctctacttcttcgagagaatactcgggaagttgatcggcgatgacagcttcgcgattccgttctggaactgggacgcccctgacgggatgcgattgccagcgatgtacgtggatcccacgtcgccgctttacgatcccctaagggatgcacagcatcagccgccgacgttagtggatttggacttcggagggatggatccttctttcagtgataagcagcagattgatcacaacctcaaggttatgtacaggcagatcgtctcgaatgcaccgacaccgaggctcttcttcggaaacccctaccgagccggcgacaatccgaaccccggtggcggctcgcttgagaacgtcccccacggaccggtccacgtctggaccggcgaccgcagccagtcggaactggaggacatgggcaacctgtactccgccgctcgcgaccccgtcttctttgcccaccactccaacatcgaccgcatctggaacgtgtggaagggtctcggtagccggcgcaaggacctggccgaccccgactggctcgacgcctccttcgtcttctacgacgagaacgccaacctcgtcaagatccgaattcgcgactgcatcgactcagacaagctacgctacgagtaccaggacgtcggtaacccatggctcaacacacgcccgacggtgacgtccggagtgaggccgagagtggccggagtggcgcatgcaaacgtcgtggagccgaagtttccgataaagttggactcggtggtgactgccaaggtgaagaggccaaaggcggcgaggaccaaggaggagaaggaggagaaggaggaggtgctggtggttgaagggatcgagctggatcgagacgtgcacgtcaaattcgacgtgttcgtgaacgtgaccgaccacgggaaggtcgggccggggggccgggagctcgccgggagcttcgtgaacgtgcctcacaggcacaagcatgacaagatgagcaagcggctgaagaccaggctgcagctgggcttgactgagctgttggaggatctcaaggctgaaggagatgggagcatcatggtgactttggtgccgaggcaggggaaggggaaggtgaaggttggcagtctcaagatcgagttagttgattga

SEQ ID NO: 16 - protein, 97 aa, synthetic construct
MASISQLIITSIPTTSCPFSPPRTTVSISGSKHHHRVSPISCSTREPQPTPRRSHSRPPPRPRRTRQPLRRLRCTNVPPRGQCGTDRGARPVRMRAG

SEQ ID NO: 17 - protein, 67 aa, synthetic construct
MASISQLITTSIPTTFPLSSSCPFSPPRTTVSISGFKHHHHVSPISCSSTTNPSLIPQSIAATSSSD

SEQ ID NO: 18 - protein, 132 aa, Musa acuminata
MASISQLITTSIPTTFSLSYSCPFSPPRTTVSISGSKHHHHVSPISCSTRDHNQPLVDPTVDRRHVLVGLGSLYGASAALTSLREASAAPIAAPDLSACGPADLPPDATPTNCCPPSAGDATEFVIPDRPRP

(19) United States
(12) Plant Patent Application Publication
(10) Pub. No.: US 2026/0206662 P1
(43) Pub. Date: Jul. 16, 2026

(54) BANANA PLANT NAMED 'POMONA'

(50) ;

(71) Applicant: Tropic Biosciences UK Limited, Norwich (GB)

(72) Inventors: David Guillaume-Schoepfer, Norwich (GB); Cristina Pignocchi, Norwich (GB)

(73) Assignee: Tropic Biosciences UK Limited, Norwich (GB)

(21) Appl. No.: 19/422,730

(22) Filed: Dec. 17, 2025

(30) Foreign Application Priority Data
Jan. 10, 2025 (GB) 2500286.6

(51) Int. Cl. A01H 6/00; A01H 5/08

(52) U.S. Cl. CPC A01H 6/00; A01H 5/08
USPC PLT/160

(57) ABSTRACT

A new banana cultivar ‘Pomona’ is provided that exhibits reduced browning of banana fruit flesh after damage or exposure to air as compared to its wildtype parent Cavendish Grande Naine. Other than the reduced browning phenotype, Pomona appears to be essentially phenotypically identical to Cavendish Grande Naine.

Latin name of the genus and species of the plant claimed: Musa acuminata Colla.

Variety denomination: Pomona.

### CROSS-REFERENCE TO RELATED APPLICATIONS

This application claims benefit of United Kingdom Patent Application No. GB 2500286.6, filed on Jan. 10, 2025, the contents of which are incorporated by reference herein in its entirety.

### BACKGROUND OF THE INVENTION

The present invention relates to a new and distinct variety of triploid (AAA) banana plant named ‘Pomona.’ The new plant was generated by using gene-editing techniques to induce mutations in three POLYPHENOL OXIDASE 1 (PPO1) genes (e.g., SEQ ID NOs: 1 to 6) in the parent variety Cavendish Grande Naine (also spelled Grand Nain; not patented). Pomona is distinguished from Cavendish Grande Naine (“Grande Naine”) primarily by a statistically significant (e.g., p-value<0.05) reduction in the browning of fruit flesh after damage or exposure to air.

### BRIEF SUMMARY OF THE INVENTION

Pomona was obtained by generating targeted edits in three PPO1 genes in Grande Naine using the CRISPR/Cas9 gene editing system. See, for example, Jiang and Doudna, “CRISPR-Cas9 Structures and Mechanisms,” Annu. Rev. Biophys., 46:505-529 (2017) for a general review of CRISPR/Cas9.

Grande Naine embryogenic cell suspension (ECS) cultures were obtained from immature male flowers or shoot tips as described in Ma, Proceedings of Symposium on Tissue Culture of Horticultural Crops, Taipei, Taiwan 8-9 Mar. 1988, pages 181-188 and/or Schoofs, “The origin of embryonic cells in Musa,” Ph.D. Thesis, KULeuven, Belgium (1997).

A binary plasmid (pMOL_0019; FIG. 1) comprising four transcriptional units within its T-DNA region (FIG. 2) was introduced to Agrobacterium tumefaciens. The four transcriptional units included sequences encoding: the marker gene nptIl, which confers resistance to certain antibiotics; human codon-optimized Streptococcus pyogenes Cas9 RNA-guided nuclease; and two single guide RNAs (sgRNAs) designed to target PPO1 genes (SEQ ID NOs: 1 to 3), while minimizing the potential for off-target editing.

The Grande Naine ECS were co-cultured with the A. tumefaciens comprising the binary plasmid as described in Khanna et al., “Centrifugation Assisted Agrobacterium tumefaciens-mediated Transformation (CAAT) of embryonic cell suspensions of banana (Musa spp. Cavendish AAA and Lady finger AAB),” Molecular Breeding, 14:239-252 (2004) and Tripathi et al., “Efficient regeneration and transformation of plantain cv. “Gonja manjaya” (Musa spp. AAB) using embryonic cell suspensions,” In Vitro Cellular & Developmental Biology—Plant, 48:216-224 (2012).

Following the co-culturing of the Grande Naine ECS and A. tumefaciens, banana cells were resuspended in 250 milliliter (mL) Erlenmeyer flasks in liquid proliferation medium with a selection agent (G418 Sulfate; Geneticin™) for up to five days with gentle shaking to produced modified ECS having mutations in PPO1 genes. The modified ECS were then washed four times in liquid proliferation medium to remove the selection agent.

Next, the modified ECS were cultured on proliferation medium, followed by embryo development medium, and then germination medium. Young shoots were then transferred to propagation medium for plantlet development. See Strosse et al. “Banana and plantain embryogenic cell suspensions,” (A. Vézina and C. Picq, eds.) INIBAP Technical Guidelines 8, The International Network for the Improvement of Banana and Plantain, Montpellier, France (2003) for more information regarding the media and protocols discussed in this paragraph.

Targeted gene edits in the three PPO1 genes were identified by extracting genomic DNA from modified banana plants and performing gene-specific polymerase chain reaction (PCR). The PCR products were cloned into vectors and subjected to Sanger sequencing methods to obtain the modified PPO1 sequences (SEQ ID NOs: 10 to 15).

Quantitative PCR (qPCR) was used to confirm that the binary plasmid sequences were absent from the modified banana plants.

Following asexual propagation of banana plant clones having mutations in the three PPO1 genes, whole genome sequencing was performed to confirm the presence of targeted edits (e.g., SEQ ID NOs: 10 to 13) and the absence of binary plasmid integration into the genome of the newly created Pomona variety.

As compared to wildtype Grande Naine, Pomona banana plants possess a single nucleotide insertion in a first PPO1 gene (SEQ ID NOs: 10 and 13), a seven nucleotide deletion and a separate single nucleotide deletion in a second PPO1 gene (SEQ ID NOS: 11 and 14), and a single nucleotide deletion in a third PPO1 gene (SEQ ID NOs: 12 and 15). All three mutations result in premature stop codons, and thus the PPO1 proteins in Pomona (SEQ ID NOs: 16 to 18) are truncated as compared to wildtype Grande Naine PPO1 proteins (SEQ ID NOs: 7 to 9). As determined by analysis of whole genome sequencing data, the binary plasmid sequences are absent from the genome of Pomona banana plants.

Pomona plants are asexually propagated via in vitro propagation techniques commonly used in the art or via corms or rhizomes to produce progeny identical to the original Pomona plant in all distinguishing characteristics.

Pomona plants are believed to exhibit phenotypes typical of Grande Naine, except Pomona banana plants exhibit a reduced browning phenotype of the banana fruit flesh after damage or exposure to air.

A homogenization assay was performed to compare the banana fruit flesh browning of Pomona and Grande Naine after damage and exposure to air.

For this assay, the ends of respective banana fruits were removed and the fruits were peeled. A 50±1 gram section of banana fruit flesh was weighed and cut into smaller cross-section slices with a scalpel to form circular discs approximately 1.5 centimeters (cm) in diameter. The peeled banana fruit slices were added to 100 mL of deionized water and pureed in a blender for 30 seconds to homogenize the samples.

Approximately 25 mL of each homogenized banana sample was transferred to a 94 millimeter Petri dish using an electric pipettor. The homogenized banana samples were then transferred to a lightbox (Duculus Light Box 2 LEDs 5500K) to be photographed from a height of 24 cm.

Within the frame of each photograph of homogenized banana sample was a reference color chart comprising seven colors ranging from light yellow to brown. Table 1 provides the color values used on the reference color chart. FIGS. 3 and 4 depict the reduced browning of Pomona as compared to Grande Naine at various time points after homogenization and the reference color chart.

TABLE 1
Color values of red (R), green (G), and blue (B) for
the seven standard colors on the reference color chart
photographed with the homogenized banana samples.

| Standard | R | G | B |
|---|---|---|---|
| 1 | 127 | 96 | 0 |
| 2 | 189 | 142 | 0 |
| 3 | 251 | 187 | 0 |
| 4 | 255 | 204 | 58 |
| 5 | 255 | 219 | 120 |
| 6 | 255 | 235 | 182 |
| 7 | 255 | 252 | 244 |

The photographs of the homogenized banana samples and reference color chart were processed in ImageJ software. See Schneider et al., Nature Methods, 9(7):671-675 (2012). Processing was performed as follows: the mean gray value (the sum of pixels' gray values in the selected area of the image divided by the number of pixels) was measured across the homogenized banana samples and in the darkest (Standard 1) and brightest (Standard 7) colors in the reference color chart. RGB browning intensity (255 minus the mean gray value) of the homogenized banana samples is normalized to that of the reference colors using the following formula: ((Sample×160)÷(Standard 1-Standard 7))−((Standard 1×160)÷(Standard 1-Standard 7))+181. The RGB browning intensity of Pomona and Grande Naine at various time points are plotted in FIG. 5 (0 minutes to 60 minutes) and FIG. 6 (0 hours to 24 hours), which also show the statistical significance (as a p-value) calculated by linear mixed modeling of repeated measurements followed by pairwise comparisons between Pomona (the test line) and Grande Naine (the control line).

Additionally, Pomona and Grande Naine fruits were peeled, cut into slices, and allowed to sit at fridge temperature (for example, at 4° C.) for up to 12 days, being photographed every other day (FIG. 7); as well as for 12 hours at room temperature (for example, at 21° C.) before being photographed, in a separate experiment (FIG. 8). The fruit flesh of Pomona was predominately non-brown, while the fruit flesh of Grande Naine turned predominately brown. Peeled and sliced banana fruit flesh from Pomona also exhibited less browning than peeled and sliced banana fruit flesh from Grande Naine up to 12 days under fridge temperature (for example, at 4° C.) when mixed with other fruits (e.g., strawberry, kiwi, blueberry, and cantaloupe melon) (FIG. 9).

A Pomona banana plant, a Pomona banana bunch, and a Pomona banana hand are shown in FIG. 10, FIG. 11, and FIG. 12, respectively.

### BRIEF DESCRIPTION OF THE DRAWINGS

FIG. 1 depicts a map of the binary vector pMOL_0019 used to generate Pomona. UTR refers to untranslated region; sgRNA refers to single guide RNA; rep refers to replication origin.

FIG. 2 depicts a map of the T-DNA element (e.g., the sequences between the Left Border and Right Border of pMOL_0019) used to generate Pomona. UTR refers to untranslated region; sgRNA refers to single guide RNA.

FIG. 3 depicts a photograph of Pomona and Grande Naine homogenization assay results at 0, 30, and 60 minutes, along with reference color charts.

FIG. 4 depicts a photograph of Pomona and Grande Naine homogenization assay results at 0 hours, 1 hour, 4 hours, and 24 hours, along with reference color charts.

FIG. 5 depicts a graph showing the statistically significant reduction in browning in Pomona (Gene-edited line) as compared to Grande Naine as determined by homogenization assays at 0 minutes (T0), 30 minutes (T30), and 60 minutes (T60). The errors bars represent one standard error.

FIG. 6 depicts a graph showing the statistically significant reduction in browning in Pomona (Gene-edited line) as compared to Grande Naine as determined by homogenization assays at 0 hours, 1 hour, 4 hours, and 24 hours. The errors bars represent one standard error.

FIG. 7 depicts photographs showing peeled and cut banana fruit flesh from Pomona and Grande Naine left at fridge temperature (for example, at 4° C.) for up to 12 days.

FIG. 8 depicts a photograph showing peeled and cut banana fruit flesh left at room temperature (for example, at 21° C.) for 12 hours from Pomona and Grande Naine.

FIG. 9 depicts sliced banana fruit flesh from Grande Naine and Pomona mixed with other fruits and left at fridge temperature (for example, at 4° C.) for up to 12 days.

FIG. 10 depicts a Pomona banana plant.

FIG. 11 depicts a Pomona banana bunch.

FIG. 12 depicts a Pomona banana hand.

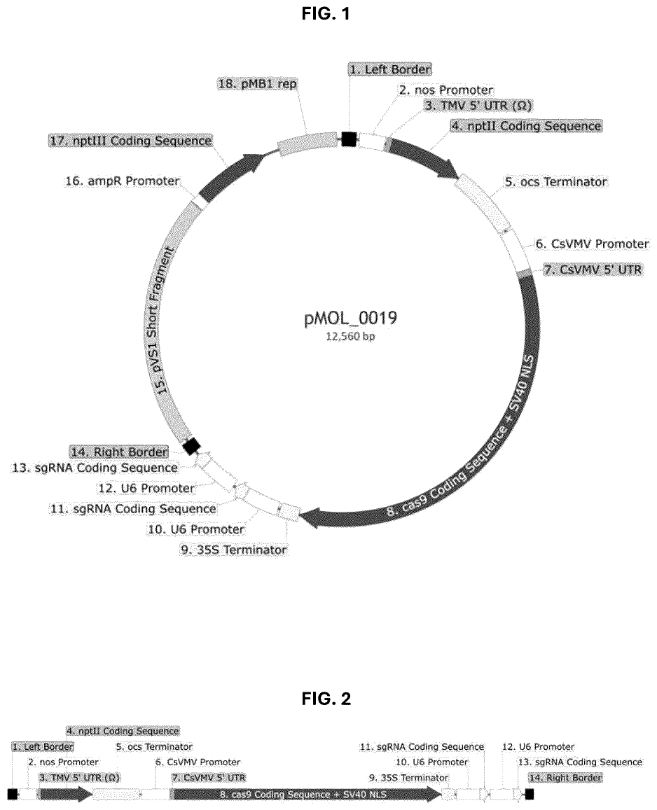

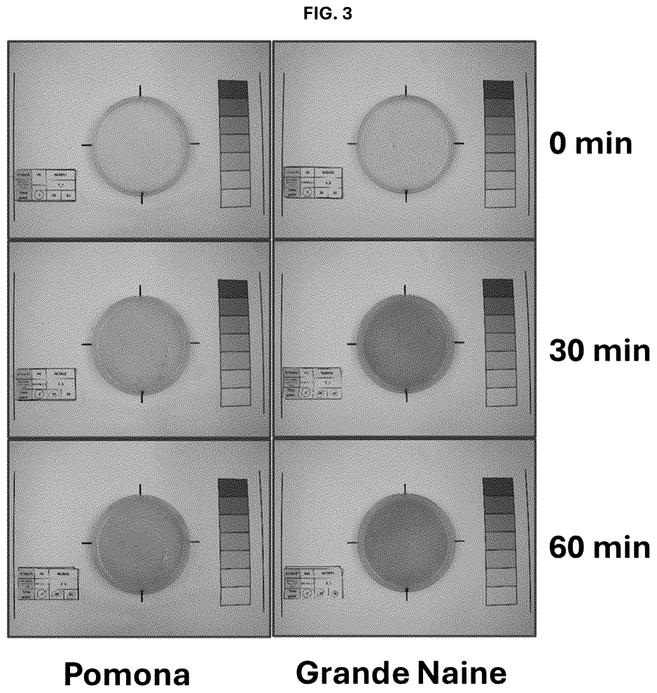

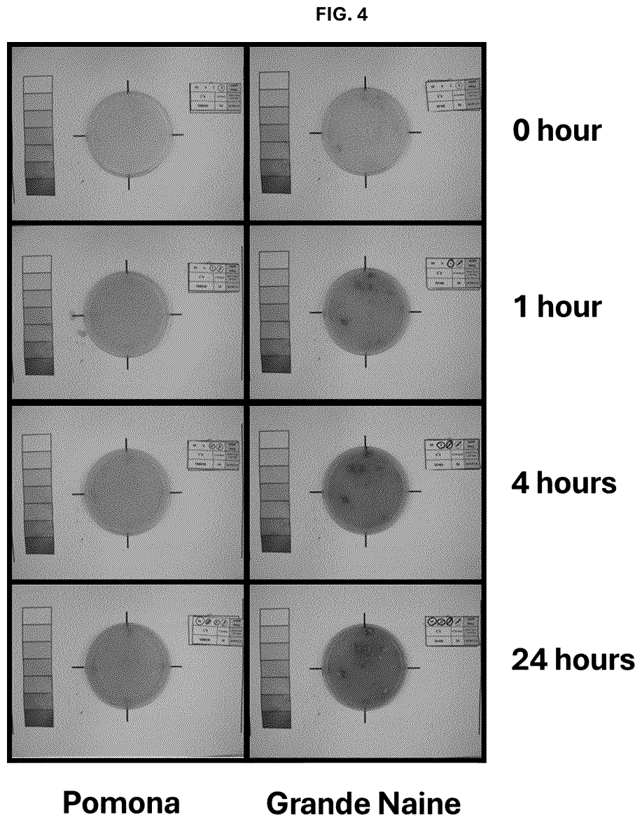

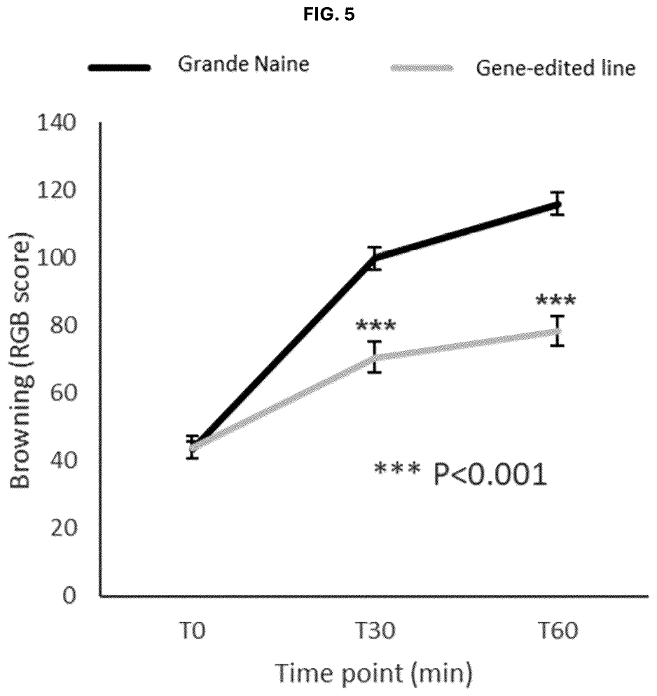

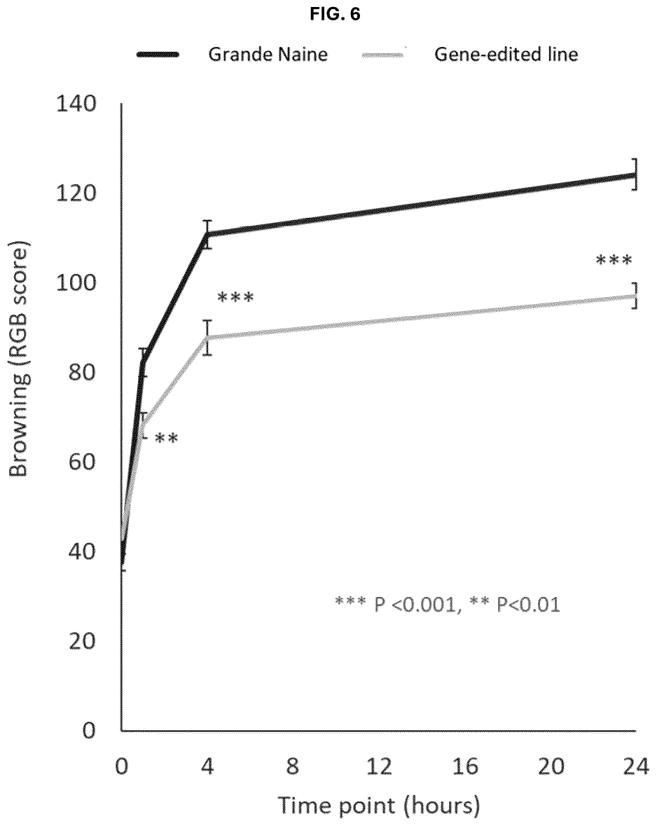

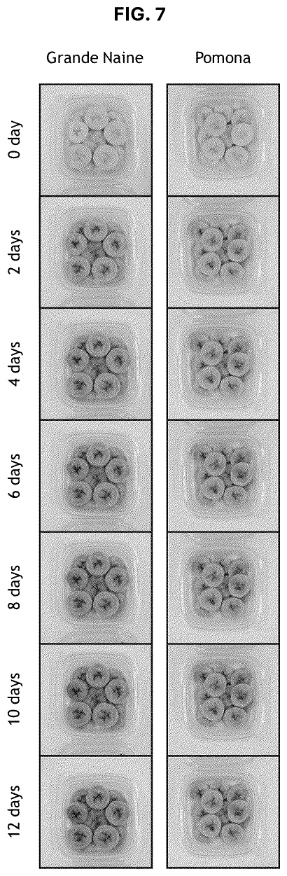

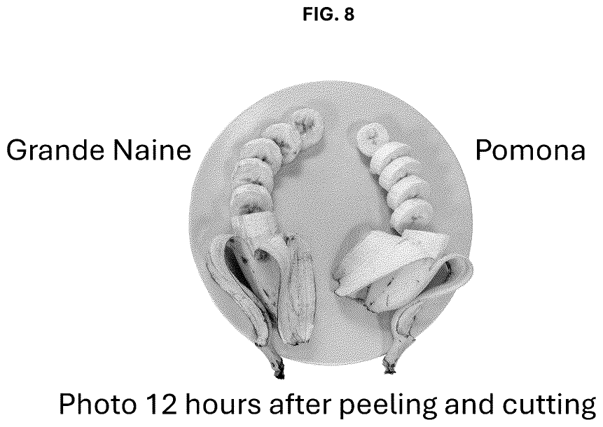

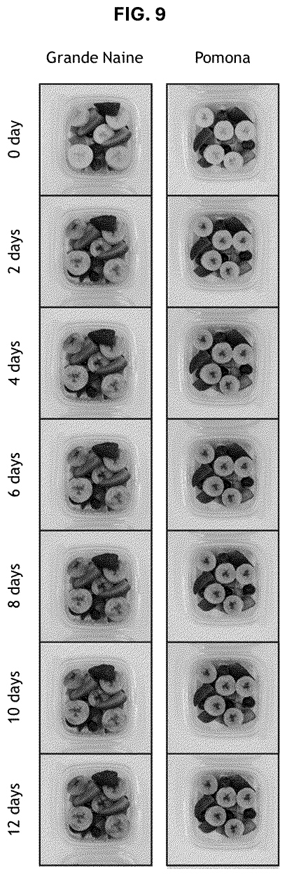

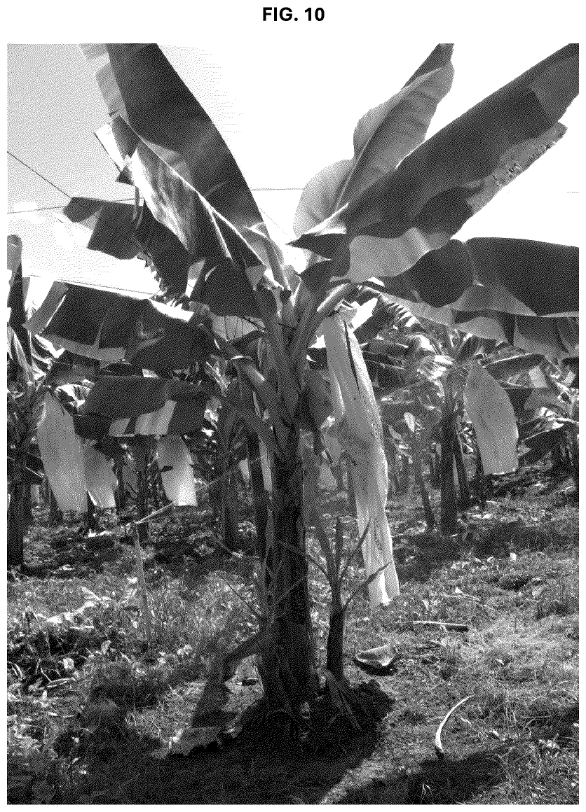

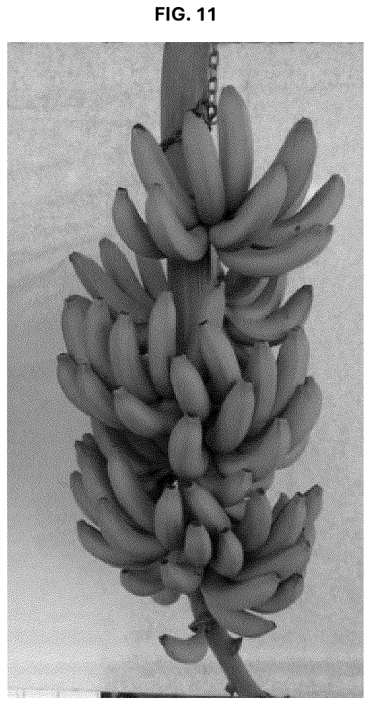

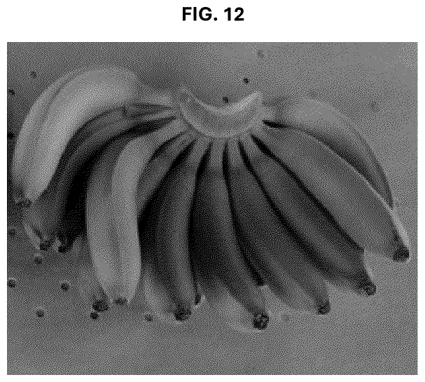

### INCORPORATION OF SEQUENCE LISTING

A sequence listing contained in the file named “V4340US-A Sequence Listing.xml” which is 45,056 bytes (measured in MS-Windows®) and created on Jun. 5, 2025, and containing 18 sequences from SEQ ID NO: 1 to SEQ ID NO: 18 is filed electronically herewith and incorporated by reference in its entirety.

Table 2 provides a description of the sequences provided in the sequence listing.

TABLE 2
Brief description of sequences.

| SEQ | Sequence | |
|---|---|---|
| ID NO | Type | Description |
| 1 | NT | Wildtype PPO1 gene genomic sequence |
| | | from a first chromosome |
| 2 | NT | Wildtype PPO1 gene genomic sequence |
| | | from a second chromosome |
| 3 | NT | Wildtype PPO1 gene genomic sequence |
| | | from a third chromosome |
| 4 | NT | Wildtype PPO1 gene CDS from a first |
| | | chromosome |
| 5 | NT | Wildtype PPO1 gene CDS from a second |
| | | chromosome |
| 6 | NT | Wildtype PPO1 gene CDS from a third |
| | | chromosome |
| 7 | AA | Wildtype PPO1 protein from a first |
| | | chromosome |
| 8 | AA | Wildtype PPO1 protein from a second |
| | | chromosome |
| 9 | AA | Wildtype PPO1 protein from a third |
| | | chromosome |
| 10 | NT | Pomona PPO1 gene genomic sequence |
| | | from a first chromosome |
| 11 | NT | Pomona PPO1 gene genomic sequence |
| | | from a second chromosome |
| 12 | NT | Pomona PPO1 gene genomic sequence |
| | | from a third chromosome |
| 13 | NT | Pomona PPO1 gene CDS from a first |
| | | chromosome |
| 14 | NT | Pomona PPO1 gene CDS from a second |
| | | chromosome |
| 15 | NT | Pomona PPO1 gene CDS from a third |
| | | chromosome |
| 16 | AA | Pomona PPO1 protein from a first |
| | | chromosome |
| 17 | AA | Pomona PPO1 protein from a second |
| | | chromosome |
| 18 | AA | Pomona PPO1 protein from a third |
| | | chromosome |

NT refers to nucleotide; AA refers to amino acid; CDS refers to coding sequence.
Wildtype refers to Cavendish Grande Naine.

### DETAILED DESCRIPTION

The following detailed description of Pomona is based on observations of plants that are approximately 9 months after planting. The plants were observed growing in a private and secure field trial site.

Certain characteristics of Pomona, such as growth and vegetative (e.g., leaf, stem) color, may change with changing environmental conditions (e.g., without being limiting, light, temperature, water availability, nutrient availability). Those of ordinary skill in the art also recognize that minor sequence changes that do not significantly alter visible plant phenotypes (e.g., silent mutations) may occur spontaneously in the genome of Pomona. Color descriptions and other terminology are used in accordance with their ordinary dictionary definitions unless the context clearly indicates otherwise.

### Botanical Description

Scientific name: Musa acuminata Cavendish ‘Pomona’.

Parentage: Cavendish Grande Naine.

Except for the reduced browning of banana fruit flesh after exposure to air or damage phenotype described above, Pomona appears to be essentially phenotypically identical to the wildtype parent Cavendish Grande Naine (unpatented). See Table 3.

TABLE 3
Phenotypic comparison of Pomona with its wildtype parent
Cavendish Grande Naine.

| | | Cavendish |
|---|---|---|
| Characteristic | Pomona | Grande Naine |
| Ploidy | Triploid | Triploid |
| Rhizome: number of | Medium | Medium |
| suckers above ground | | |
| Pseudostem: length | Medium | Medium |
| Pseudostem: diameter | Medium | Medium |
| Pseudostem: overlapping | Strong | Strong |
| of leaf sheaths | | |
| Pseudostem: Tapering | Absent or Weak | Absent or weak |
| Plant: growth habit | Spreading | Spreading |
| Petiole: attitude of wings | Curved outwards | Curved outwards |
| at base | | |
| Petiole: Length | Medium | Medium |
| Leaf blade: colour of | Green | Green |
| midrib on lower side | | |
| Leaf blade: shape of base | Both sides acute | Both sides acute |
| Leaf blade: length | Long | Long |
| Leaf blade: width | Broad | Broad |
| Leaf blade: ratio length/ | Moderately | Moderately |
| width | elongated | elongated |
| Leaf blade: glossiness of | Absent | Absent |
| upper side | | |
| Peduncle: length | Medium | Medium |
| Peduncle: diameter | Large | Large |
| Peduncle: pubescence | Absent | Absent |
| Bunch: length | Long | Long |
| Bunch: diameter | Broad | Broad |
| Bunch: shape | Cylindrical | Cylindrical |
| Bunch: attitude of fruits | Moderately turned | Moderately turned |
| | up | up |
| Bunch: compactness | Medium | Medium |
| Bunch: number of hands | Many | Many |
| Rachis: attitude of male | Vertical | Vertical |
| part | | |
| Rachis: prominence of | Weak | Weak |
| scars | | |
| Rachis: persistence of | Absent or weak | Absent or weak |
| bracts | | |
| Fruit: length | Medium | Medium |
| Fruits: length of pedicel | Medium | Medium |
| Fruit: persistence of | Absent | Absent |
| floral organs | | |
| Male inflorescence: | Present | Present |
| persistence | | |
| Male inflorescence: | Narrow ovate | Narrow ovate |
| shape | | |
| Male inflorescence: | Closed or slight | Closed or slight |
| opening of bracts | open | open |
| Bract: shape of apex | Broad acute | Broad acute |
| Fruit: Color of Flesh | Slow browning | Rapid browning |
| after exposure to air | after banana fruit | after banana fruit |
| | flesh damage and/ | flesh damage and/ |
| | or exposure to air; | or exposure to air; |
| | for example, | for example, |
| | predominately | predominantly |
| | non-brown after | brown 12 hours at |
| | 12 hours at room | room temperature |
| | temperature | following peeling |
| | following peeling | and cutting |
| | and cutting | |

We claim:

1. A new and distinct variety of banana plant, substantially as herein shown and described.
